(19) United States
(12) Patent Application Publication
(10) Pub. No.: US 2023/0377756 A1
(43) Pub. Date: Nov. 23, 2023

(54) CONSTRUCTION METHOD OF SURVIVAL PREDICTION MODEL FOR HEPATOCELLULAR CARCINOMA PATIENT BASED ON CELL DEATH-RELATED GENES

(71) Applicant: The First Affiliated Hospital of Sun Yat-Sen University, Guangzhou (CN)

(72) Inventors: Jiaping Li, Guangzhou (CN); Guixiong Zhang, Guangzhou (CN)

(21) Appl. No.: 18/313,751

(22) Filed: May 8, 2023

(30) Foreign Application Priority Data
May 20, 2022 (CN) 202210559294.6

(51) Int. Cl. G16H 50/30; G16H 50/20; G16B 25/10

(52) U.S. Cl. CPC G16H 50/30; G16H 50/20; G16B 25/10

(57) ABSTRACT

The present disclosure specifically provides a construction method of a survival prediction model for hepatocellular carcinoma patients based on cell death-related genes, including the following steps of: S1, constructing a preliminary survival risk score prediction model for hepatocellular carcinoma patients; S2, using a public database TCGA as a training set, and expressing genes based on the basis of differential expression related to three novel programmed cell death pathways including cell autophagy, cell ferroptosis and cell pyroptosis; S3, determining genes related to the lifetime through single-factor Cox regression analysis; S4, screening the genes related to the lifetime through multi-factor Cox regression analysis, and training to obtain a final survival risk score prediction model for hepatocellular carcinoma patients; and S5, calculating a risk index based on the gene related expression quantity and the risk related coefficient, and analyzing and performing external verification. According to the method, the survival of hepatocellular carcinoma patients can be accurately predicted based on a prognosis model of three novel programmed cell death-related genes, and a new direction is provided for the diagnosis and treatment of hepatocellular carcinoma.

### TECHNICAL FIELD

The present disclosure relates to the field of medical technologies, and in particular to a construction method of a survival prediction model for hepatocellular carcinoma patients based on cell death-related genes.

### BACKGROUND

Primary liver cancer is the sixth most common malignant tumor and is the third leading cause of cancer death. Hepatocellular carcinoma accounts for 75% of 85% of primary liver cancer cases. However, due to a limited number of available biomarkers and a complex etiology and high heterogeneity of hepatocellular carcinoma, predicting the prognosis of hepatocellular carcinoma patients remains challenging. Cell autophagy, cell ferroptosis and cell pyroptosis are the new patterns of programmed cell death discovered in recent years. These three novel types of programmed cell death are crucial in the occurrence and progression of hepatocellular carcinoma. However, it is still unknown whether the expression of three novel programmed cell death-related genes is related to hepatocellular carcinoma patients' survival and prognosis.

### SUMMARY

The present disclosure provides a construction method of a survival prediction model for hepatocellular carcinoma patients based on cell death-related genes, in order to overcome the problems raised in the background art.

A construction method of a survival prediction model for hepatocellular carcinoma patients based on cell death-related genes, including the following steps of:

S1, developing a preliminary survival risk score prediction model for hepatocellular carcinoma patients.

S2, using a public database TCGA as a training set, and expressing genes based on differential expression related to three novel programmed cell death pathways including cell autophagy, cell ferroptosis and cell pyroptosis;

S3, identifying genes associated with longevity using single-factor Cox regression analysis and clinical data from the TCGA database of of patients with hepatocellular carcinoma;

S4, using multi-factor Cox regression analysis to identify genes for constructing a risk scoring model, and then inputting the screened genes into a preliminary survival risk score prediction model for hepatocellular carcinoma patients and training to obtain a final survival risk score prediction model for hepatocellular carcinoma patients; and

S5, calculating a risk index according to the gene-related expression quantity and a risk-related coefficient, analyzing the survival prediction of the database TCGA by using the risk index and performing external verification through another public database, ICGC as a verification set.

As a preferred embodiment, in S4, the genes related to the lifetime through multi-factor Cox regression analysis include five cell autophagy genes, three cell ferroptosis genes and two cell pyroptosis genes.

As a preferred embodiment, the five cell autophagy genes are BIRC5, SQSTM1, HDAC1, RHEB and ATIC, respectively.

As a preferred embodiment, the three cell ferroptosis genes are G6PD, ACACA and SLC1A5, respectively.

As a preferred embodiment, the two cell pyroptosis genes are BAK1 and GSDME, respectively.

As a preferred embodiment, the risk index=0.1450955×BIRC5 gene expression level+0.19642991×SQSTM1 gene expression level+0.37106235×HDAC gene expression level+0.3770679×RHEB gene expression level+0.34668129×ATIC gene expression level+0.16196511×G6PD gene expression level+0.4,035,343×ACACA gene expression level+0.20555184×SLC1A5 gene expression level+0.28470975×BAK1 gene expression level+0.44820065×GSDME gene expression level.

As a preferred embodiment, patients in a TCGA queues are divided into a high-risk group and a low-risk group based on a median risk index value, and a Kaplan-Meier survival curve is drawn according to independent clinical factors obtained from the Cox regression analysis combined with the risk score prediction model.

As a preferred embodiment, the prediction performance of the risk index on a total survival time is evaluated through a time-dependent ROC curve.

As a preferred embodiment, the area under the Kaplan-Meier survival curve in one year, two years and three years is calculated, respectively.

As a preferred embodiment, patients in an ICGC queues are divided into a high-risk group and a low-risk group based on the median risk index value, and analysis results on the survival prognosis of TCGA are verified by results of the ICGC queues.

Beneficial effects: the present disclosure provides a construction method of a survival prediction model for hepatocellular carcinoma patients based on cell death-related genes. According to the method, the survival of hepatocellular carcinoma patients can be accurately predicted based on a prognosis model of three novel programmed cell death-related genes, and a new direction and strategy are provided for the grouping and hierarchical management and precise treatment of hepatocellular carcinoma. The risk index can be used as an independent prognostic factor to predict the survival of patients with hepatocellular carcinoma, and another public database, ICGC, is used as the validation set to externally verify the universal applicability of the model, providing a new direction for the hepatocellular carcinoma diagnosis and treatment.

### BRIEF DESCRIPTION OF THE DRAWINGS

FIG. 1 shows a step diagram of a flow of the present disclosure.

FIG. 2 shows a specific illustration diagram of an embodiment of the present disclosure.

FIG. 3 shows a total survival time and area under the curve (AUC) of a low-risk group in the survival curves of TCGA and ICGC queues in 1, 2 and 3 years, respectively.

FIG. 4 shows a survival curve of a disease-free survival in TCGA and GSE14520 queues.

### DETAILED DESCRIPTION OF THE EMBODIMENTS

The present disclosure will now be detailed with reference to the accompanying drawings. These figures are simplified schematics that only illustrate the basic structure of the present disclosure and only show the composition related to the present disclosure. For those skilled in the art, the specific meanings of the above terms in the present disclosure can be understood in accordance with specific cases.

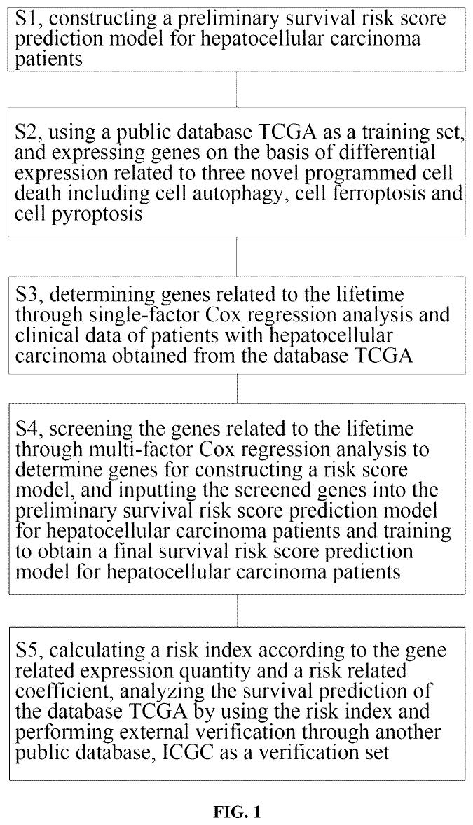

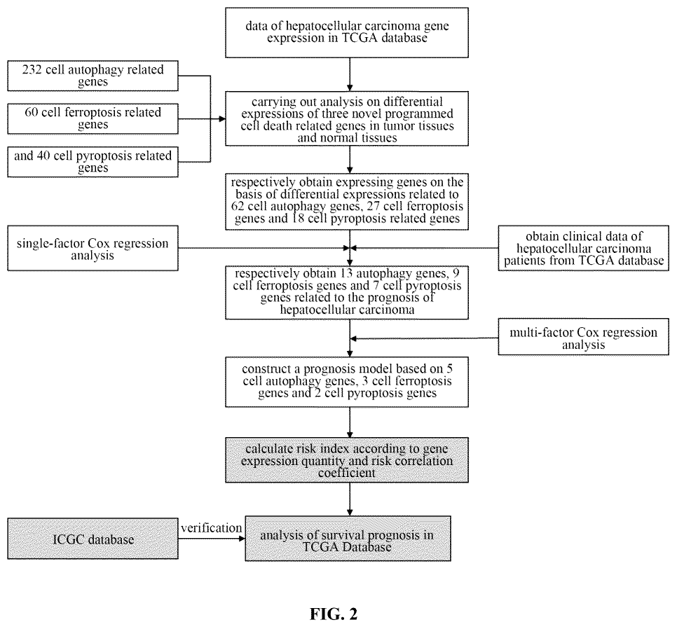

As shown in FIGS. 1 and 2, the present disclosure provides a construction method of a survival prediction model for hepatocellular carcinoma patients based on cell death-related genes, including the following steps:

S1, constructing a preliminary survival risk score prediction model for hepatocellular carcinoma patients;

S2, using a public database TCGA as a training set that includes 232 cell autophagy-related genes, 60 cell ferroptosis-related genes and 40 cell pyroptosis-related genes, and carrying out analysis on differential expressions of the above three novel programmed cell death-related genes in tumor tissues and normal tissues to obtain expressing genes on the basis of differential expressions related to 62 cell autophagy genes, 27 cell ferroptosis genes and 18 cell pyroptosis related genes;

S3, determining genes related to the lifetime through single-factor Cox regression analysis and clinical data of patients with hepatocellular carcinoma obtained from the database TCGA, wherein 13 autophagy genes, 9 cell ferroptosis genes and 7 cell pyroptosis genes related to the prognosis of hepatocellular carcinoma are obtained; respectively;

S4, screening the genes related to the lifetime through multi-factor Cox regression analysis to determine genes for constructing a risk score model, with the screened genes consisting of 5 autophagy genes, 3 cell ferroptosis genes and 2 cell pyroptosis genes, and inputting the screened genes into the preliminary survival risk score prediction model for hepatocellular carcinoma patients and training to obtain a final survival risk score prediction model for hepatocellular carcinoma patients; and

S5, calculating a risk index according to the gene-related expression quantity and the risk-related coefficient, analyzing the survival prediction of the database TCGA by using the risk index and performing external verification through another public database, ICGC as a verification set.

In a specific example of the present disclosure, the 5 cell autophagy genes are BIRC5, SQSTM1, HDAC1, RHEB and ATIC, respectively; the 3 screened cell ferroptosis genes are G6PD, ACACA and SLC1A5, respectively; the screened 2 cell pyroptosis genes are BAK1 and GSDME, respectively; the risk index is calculated from the relevant expression quantity and risk correlation coefficient of the above 10 genes by the following formula: the risk index=0.1450955×BIRC5 gene expression level+0.19642991×SQSTM1 gene expression level+0.37106235×HDAC gene expression level+0.3770679×RHEB gene expression level+0.34668129×ATIC gene expression level+0.16196511×G6PD gene expression level+0.4,035,343×ACACA gene expression level+0.20555184×SLC1A5 gene expression level+0.28470975×BAK1 gene expression level+0.44820065×GSDME gene expression level.

In a specific example of the present disclosure, patients in a TCGA queues are divided into a high-risk group and a low-risk group based on the median risk index value, and a Kaplan-Meier survival curve is drawn according to independent clinical factors obtained from the Cox regression analysis combined with the risk score prediction model: the prediction performance of the risk index on a total survival time is evaluated through a time-dependent ROC curve; the area under the Kaplan-Meier survival curve in 1 year, 2 years and 3 years is calculated, respectively: patients in an ICGC queues are divided into a high-risk group and a low-risk group based the median risk index value, and analysis results on survival prognosis of TCGA are verified by results of the ICGC queues.

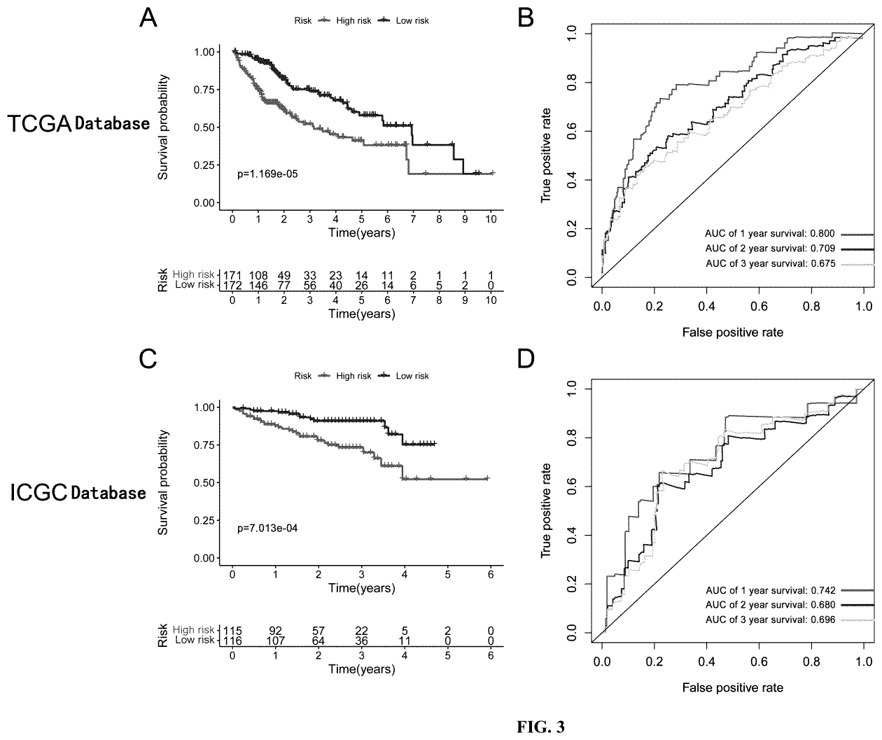

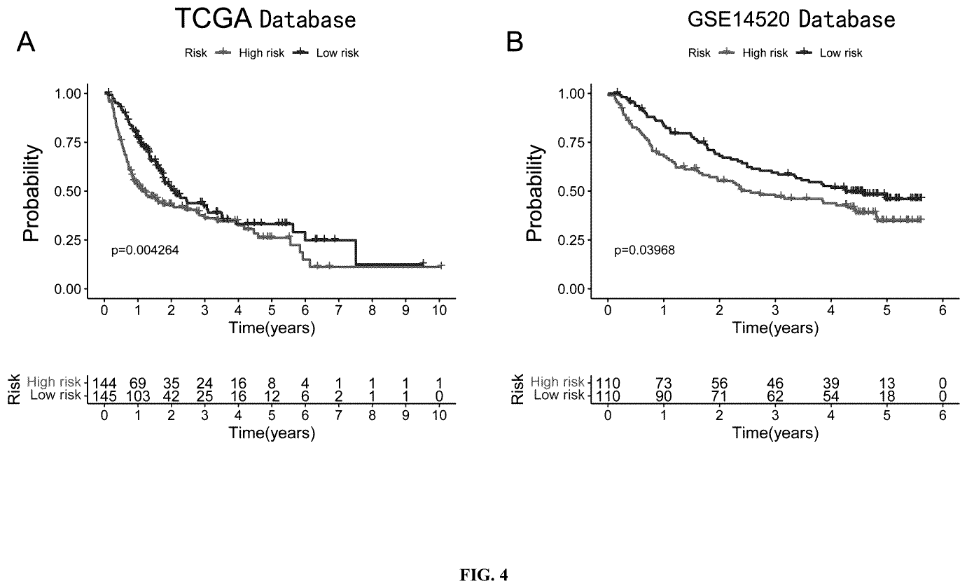

As shown in FIGS. 3 and 4, the Kaplan-Meier survival curve results based on the median risk index (RI) value show that the total survival time (OS) (FIG. 3A) and disease-free survival time (FIG. 4A and FIG. 4B) of the low-risk group are significantly longer than those of the high-risk group. The prediction performance of RI on OS is evaluated by the time-dependent ROC curve. The area under the curve (AUC) reaches 0.800, 0.709 and 0.675 in 1, 2 and 3 years, respectively (FIG. 3B). Patients in an ICGC queues are divided into a high-risk group and a low-risk group based on the median risk index value. The results of the ICGC queues are similar to those of TCGA (FIG. 3C and FIG. 3D).

The present disclosure provides a method for developing a survival prediction model for patients with hepatocellular carcinoma based on cell death-related genes. According to the method, the survival of hepatocellular carcinoma patients can be accurately predicted based on a prognosis model of three novel programmed cell death-related genes, and a new direction and strategy are provided for the grouping and hierarchical management and precise treatment of hepatocellular carcinoma. The risk index can be used as an independent prognostic factor to predict the survival of patients with hepatocellular carcinoma, and another public database, ICGC, is used as the validation set to externally verify the universal applicability of the model, providing a new direction for the diagnosis and treatment of hepatocellular carcinoma.

In the description of the present disclosure, the use of reference terms “one embodiment”, “certain embodiments”, “schematic embodiments”, “examples”, “specific examples”, or “some examples” means that specific features, structures, materials, or characteristics described in conjunction with the embodiments or examples are included in at least one embodiment or example of the present disclosure. In the description, the schematic expressions of the above terms do not always refer to the same embodiments or examples. Furthermore, the specific features, structures, materials or characteristics described may be combined in an appropriate manner in any one or more embodiments or examples.

The preceding illustrates and describes the fundamental principles, key features and advantages of the present disclosure. For those skilled in the art, it is obvious that the present disclosure is not limited to the details of the exemplary embodiments discussed above, and that it can be realized in other specific forms without departing from the spirit or basic features of the present disclosure. As a result, the embodiments should be regarded as exemplary and non-restrictive from any perspective. The scope of the present disclosure is defined by the appended claims rather than the above description. Therefore, all changes within the meaning and scope of the equivalent elements of the claims in the present disclosure are intended to be included. Any reference sign in the claims is not intended to limit the scope of the claims.

In addition, it should also be noted that while the Description is stated according to the embodiments, not every embodiment contains only an independent technical solution. This definition of the Description is only for the sake of clarity. Those skilled in the art should consider the Description as a whole, and the technical solutions in each embodiment can be properly combined to form other embodiments that can be understood by those skilled in the art.

What is claimed is:

1. A construction method of a survival prediction model for hepatocellular carcinoma patients based on cell death-related genes, comprising the following steps of:
S1, constructing a preliminary survival risk score prediction model for hepatocellular carcinoma patients;
S2, using a public database TCGA as a training set, and expressing genes on the basis of differential expression related to three novel programmed cell death including cell autophagy, cell ferroptosis and cell pyroptosis;
S3, determining genes related to the lifetime through single-factor Cox regression analysis and clinical data of patients with hepatocellular carcinoma obtained from the database TCGA;
S4, screening the genes related to the lifetime through multi-factor Cox regression analysis to determine genes for constructing a risk score model, and inputting the screened genes into the preliminary survival risk score prediction model for hepatocellular carcinoma patients and training to obtain a final survival risk score prediction model for hepatocellular carcinoma patients; and
S5, calculating a risk index according to the gene-related expression quantity and a risk-related coefficient, analyzing the survival prediction of the database TCGA by using the risk index and performing external verification through another public database, ICGC as a verification set.

2. The construction method of the survival prediction model for the hepatocellular carcinoma patients based on the cell death-related genes according to claim 1, wherein in S4, the genes related to the lifetime through multi-factor Cox regression analysis comprise five cell autophagy genes, three cell ferroptosis genes and two cell pyroptosis genes.

3. The construction method of the survival prediction model for the hepatocellular carcinoma patients based on the cell death-related genes according to claim 2, wherein the five cell autophagy genes are BIRC5, SQSTM1, HDAC1, RHEB and ATIC, respectively.

4. The construction method of the survival prediction model for the hepatocellular carcinoma patients based on the cell death-related genes according to claim 3, wherein the three cell ferroptosis genes are G6PD, ACACA and SLC1A5, respectively.

5. The construction method of the survival prediction model for the hepatocellular carcinoma patients based on the cell death-related genes according to claim 4, wherein the two cell pyroptosis genes are BAK1 and GSDME, respectively.

6. The construction method of the survival prediction model for the hepatocellular carcinoma patients based on the cell death-related genes according to claim 5, wherein the risk index=0.1450955×BIRC5 gene expression level+0.19642991×SQSTM1 gene expression level+0.37106235×HDAC gene expression level+0.3770679×RHEB gene expression level+0.34668129×ATIC gene expression level+0.16196511×G6PD gene expression level+0.4,035,343×ACACA gene expression level+0.20555184×SLC1A5 gene expression level+0.28470975×BAK1 gene expression level+0.44820065×GSDME gene expression level.

7. The construction method of the survival prediction model for the hepatocellular carcinoma patients based on the cell death-related genes according to claim 1, wherein patients in a TCGA queues are divided into a high-risk group and a low-risk group based on a median risk index value, and a Kaplan-Meier survival curve is drawn according to independent clinical factors obtained from the Cox regression analysis combined with the risk score prediction model.

8. The construction method of the survival prediction model for the hepatocellular carcinoma patients based on the cell death-related genes according to claim 7, wherein the prediction performance of the risk index on a total survival time is evaluated through a time-dependent ROC curve.

9. The construction method of the survival prediction model for the hepatocellular carcinoma patients based on the cell death-related genes according to claim 8, wherein the area under the Kaplan-Meier survival curve in 1 year, 2 years and 3 years is calculated, respectively.

10. The construction method of the survival prediction model for the hepatocellular carcinoma patients based on the cell death-related genes according to claim 9, wherein patients in an ICGC queues are divided into a high-risk group and a low-risk group based on the median risk index value, and analysis results on survival prognosis of TCGA are verified by results of the ICGC queues.
